(19) United States
(12) Patent Application Publication
(10) Pub. No.: US 2025/0312128 A1
(43) Pub. Date: Oct. 9, 2025

(54) INFERIOR ALVEOLAR NERVE INDICATION METHOD, COMPUTING DEVICE, AND COMPUTER READABLE RECORDING MEDIUM THEREFOR

(71) Applicant: OSSTEM IMPLANT CO., LTD., Seoul (KR)

(72) Inventors: Tae Hoon YONG, Seoul (KR); Hyoung Mok KIM, Seoul (KR)

(21) Appl. No.: 18/853,010

(22) PCT Filed: May 18, 2023
(86) PCT No.: PCT/KR2023/006779

§ 371 Date: Jun. 24, 2025

(30) Foreign Application Priority Data
Jun. 8, 2022 (KR) 10-2022-0069567

(51) Int. Cl. A61C 13/00; A61B 6/03; A61B 6/50; A61B 6/58; G06T 7/73; G06T 17/00; G06T 19/00; G06T 19/20

(52) U.S. Cl. CPC A61C 13/0004; A61B 6/032; A61B 6/506; A61B 6/582; G06T 7/73; G06T 17/00; G06T 19/006; G06T 19/20; G06T 2207/10081; G06T 2207/30036; G06T 2210/41; G06T 2219/004; G06T 2219/2012

(57) ABSTRACT

Disclosed are an inferior alveolar nerve indication method, a computing device, and a computer-readable recording medium therefor, and the inferior alveolar nerve indication method comprises the steps of displaying a dental image of a patient, marking an inferior alveolar nerve on the dental image according to the location of a mandibular nerve predicted from the dental image, and displaying, on the dental image, a rendered three-dimensional (3D) tube based on the inferior alveolar nerve.

### CROSS-REFERENCE TO RELATED APPLICATIONS

This application is a National Stage of International patent application PCT/KR2023/006779, filed on May 18, 2023, which claims priority of foreign Korean patent application no. KR 10-2022-0069567, filed on Jun. 8, 2022, the disclosures of which are hereby incorporated by reference in their entireties.

### TECHNICAL FIELD

The present invention relates to an inferior alveolar nerve displaying method, and more particularly, to a device and method for displaying an inferior alveolar nerve as an image in a dental imaging system.

### BACKGROUND ART

In implant surgery using software of a dental imaging system, the surgery may be performed after establishing an implant placement plan by considering an implant structure (e.g., a crown, an implant body, an abutment, and the like) and an anatomical structure of a patient on the software.

During the surgery, when planning an implant placement in a mandible, in order to prevent damage to a mandibular nerve canal (or an inferior alveolar nerve), a mandibular nerve area may be identified on a CT (computed tomography) image, and a collision with the implant structure may be predicted in advance by generating a tube-shaped inferior alveolar nerve line connecting nerves using a function provided by the software of the dental imaging system to establish a surgery plan.

However, since a conventional dental imaging system does not display a distance between the inferior alveolar nerve and an implant according to an implant placement location to an extent that a user may easily recognize the distance, there is a limitation that the user has to check a location of a nerve during an implant procedure.

In addition, the conventional dental imaging system has a limitation that it is difficult for the user to distinguish a boundary between the inferior alveolar nerve and a bone.

Thus, a method is required to guide to the location of a nerve according to an implantation depth and to allow the user to visually clearly distinguish the boundary between the inferior alveolar nerve and the bone.

### DISCLOSURE OF THE INVENTION

### Technical Goals

The present invention may provide a device and method capable of providing a location guide for a mandibular nerve during an implant placement by marking, on a three-dimensional (3D) temporary implant structure, a color indicating a distance between an inferior alveolar nerve and the 3D temporary implant structure according to an implantation depth of the 3D temporary implant structure, wherein the 3D temporary implant structure may indicate an implant body to be placed.

In addition, the present invention may provide a device and method capable of visually clearly distinguishing a boundary between a nerve and a bone by displaying a color map that assigns weights to objects according to a distance between the objects such as a mandibular nerve center point, the bone, or an implant structure, based on the mandibular nerve center point.

### Technical Solutions

An inferior alveolar nerve displaying method according to an embodiment of the present invention may include displaying a dental image of a patient; marking an inferior alveolar nerve on the dental image according to a location of a mandibular nerve predicted from the dental image; and displaying, on the dental image, a rendered three-dimensional (3D) tube based on the inferior alveolar nerve.

The marking of the inferior alveolar nerve of the inferior alveolar nerve displaying method according to an embodiment of the present invention may include detecting, using artificial intelligence (AI), mandibular nerve center points from the dental image; and generating an inferior alveolar nerve by connecting the detected mandibular nerve center points and marking the generated inferior alveolar nerve.

The displaying of the 3D tube of the inferior alveolar nerve displaying method according to an embodiment of the present invention may include generating the 3D tube by rendering a tube having a preset radius centered on a mandibular nerve center point included in the inferior alveolar nerve.

The inferior alveolar nerve displaying method according to an embodiment of the present invention may further include correcting a CT number of an object, which is an anatomical structure included in the dental image.

The correcting of the CT number of the inferior alveolar nerve displaying method according to an embodiment of the present invention may include correcting the CT number of the object in proportion to a distance between the object and the inferior alveolar nerve.

The correcting of the CT number of the inferior alveolar nerve displaying method according to an embodiment of the present invention may include setting a CT number of an inside of the inferior alveolar nerve to be lower than a CT number of an outside of the inferior alveolar nerve.

The inferior alveolar nerve displaying method according to an embodiment of the present invention may further include marking, on a 3D temporary implant structure, a color indicating a distance between the 3D temporary implant structure and the inferior alveolar nerve according to an implantation depth of the 3D temporary implant structure, wherein the 3D temporary implant structure indicates an implant body to be placed, during an implant placement.

The displaying on the 3D temporary implant structure of the inferior alveolar nerve displaying method according to an embodiment of the present invention may include placing a 3D temporary implant structure generated according to a shape of an implant to be placed in a patient in a gum area of the dental image of the patient; correcting a CT number of each of three-dimensional (3D) temporary implant structure areas in proportion to a distance between the inferior alveolar nerve and each of the 3D temporary implant structure areas that changes during a placement process; and displaying a color of each of the 3D temporary implant structure areas according to the corrected CT number.

A computing device according to an embodiment of the present invention may include a processor; and a memory configured to load or store a program executed by the processor, wherein the program may be configured to perform displaying a dental image of a patient, marking an inferior alveolar nerve on the dental image according to a location of a mandibular nerve predicted from the dental image; and displaying a three-dimensional (3D) tube based on the inferior alveolar nerve.

The computing device, wherein the processor of the computing device according to an embodiment of the present invention may detect, using artificial intelligence (AI), mandibular nerve center points from the dental image, and generate an inferior alveolar nerve by connecting the detected mandibular nerve center points and mark the generated inferior alveolar nerve.

The processor of the computing device according to an embodiment of the present invention may generate the 3D tube by rendering a tube having a preset radius centered on a mandibular nerve center point included in the inferior alveolar nerve.

The processor of the computing device according to an embodiment of the present invention may correct a CT number of an object, which may be an anatomical structure included in the dental image.

The processor of the computing device according to an embodiment of the present invention may correct a CT number of the object in proportion to a distance between the object and the inferior alveolar nerve.

The processor of the computing device according to an embodiment of the present invention may set a CT number of an inside of the inferior alveolar nerve to be lower than a CT number of an outside of the inferior alveolar nerve.

The processor of the computing device according to an embodiment of the present invention may mark, on a 3D temporary implant structure, a color indicating a distance between the 3D temporary implant structure and the inferior alveolar nerve according to an implantation depth of the 3D temporary implant structure, wherein the 3D temporary implant structure indicates an implant body to be placed, during an implant placement.

### Effects of the Invention

According to an embodiment of the present invention, an inferior alveolar nerve line and a surrounding structure may be expressed through a color map based on a location of a mandibular nerve predicted from a dental image.

In addition, according to an embodiment of the present invention, during an implant placement, a location guide for a mandibular nerve may be provided by marking, on a three-dimensional (3D) temporary implant structure, a color indicating a distance between an inferior alveolar nerve and the 3D temporary implant structure according to an implantation depth of the 3D temporary implant structure, wherein the 3D temporary implant structure may indicate an implant body to be placed.

In addition, according to an embodiment of the present invention, a boundary between a nerve and a bone may be visually clearly distinguished by displaying a color map that assigns weights to objects according to a distance between the objects such as a mandibular nerve center point, the bone, or an implant structure, based on the mandibular nerve center point.

### BRIEF DESCRIPTION OF DRAWINGS

FIG. 1 is a diagram illustrating a computing device that performs an inferior alveolar nerve displaying method, according to an embodiment of the present invention.

FIG. 2 is an example of an inferior alveolar nerve displaying process according to an embodiment of the present invention.

FIG. 3 is an example of an inferior alveolar nerve displayed according to an embodiment of the present invention.

FIG. 4 is an example illustrating a two-dimensional (2D) sagittal section of an inferior alveolar nerve according to an embodiment of the present invention.

FIG. 5 is an example illustrating a 2D coronal section of an inferior alveolar nerve according to an embodiment of the present invention.

FIG. 6 is an example illustrating a 2D axial cross section of an inferior alveolar nerve according to an embodiment of the present invention.

FIG. 7 is an example of guiding to a location of an inferior alveolar nerve during an implant placement, according to an embodiment of the present invention.

FIG. 8 is a flowchart illustrating an inferior alveolar nerve displaying method according to an embodiment of the present invention.

### BEST MODE FOR CARRYING OUT THE INVENTION

Hereinafter, embodiments are described in detail with reference to the accompanying drawings. However, various alterations and modifications may be made to the embodiments. Here, the embodiments are not meant to be limited by the descriptions of the present disclosure. The embodiments should be understood to include all changes, equivalents, and replacements within the idea and the technical scope of the disclosure.

The terminology used herein is for the purpose of describing particular embodiments only and is not intended to be limiting. The singular forms “a,” “an,” and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

When describing the examples with reference to the accompanying drawings, like reference numerals refer to like components and a repeated description related thereto is omitted. In the description of example embodiments, detailed description of well-known related structures or functions will be omitted when it is deemed that such description will cause ambiguous interpretation of the present disclosure.

Hereinafter, embodiments are described in detail with reference to the accompanying drawings.

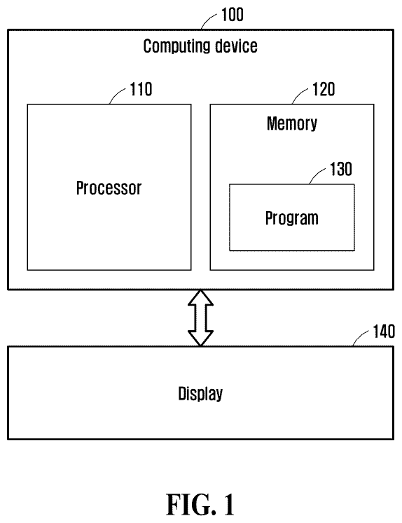

FIG. 1 is a diagram illustrating a computing device that performs an inferior alveolar nerve displaying method, according to an embodiment of the present invention.

Referring to FIG. 1, a computing device 100 may include at least one processor 110 and a memory 120 for loading or storing a program 130 performed by the processor 110. The components included in the computing device 100 of FIG. 1 are only an example, and one of ordinary skill in the art to which the present invention pertains may understand that other generally used components may further be included in addition to the components shown in FIG. 1.

The processor 110 may control an overall operation of each component of the computing device 100. The processor 110 may be configured by including a central processing unit (CPU), a microprocessor unit (MPU), a microcontroller unit (MCU), a graphics processing unit (GPU), or other well-known types of processors in the art of the present invention. In addition, the processor 110 may perform an operation of at least one application or program to perform the methods/operations described herein according to embodiments. The computing device 100 may include at least one processor.

The memory 120 may store one of, or a combination of two or more of, various pieces of data, instructions, and pieces of information that are used by a component (e.g., the processor 110) included in the computing device 100. The memory 120 may include a volatile memory or a non-volatile memory.

The program 130 may include one or more actions through which the methods/operations described herein according to embodiments are implemented and may be stored in the memory 120 as software. In this case, an operation may correspond to a command that is implemented in the program 130. For example, the program 130 may include an operation of displaying a dental image of a patient, an operation of marking inferior alveolar nerve according to a location of a mandibular nerve predicted from the dental image, and an operation of displaying a rendered three-dimensional (3D) tube based on the inferior alveolar nerve.

When the program 130 is loaded in the memory 120, the processor 110 may perform methods/operations according to various embodiments of the present invention by executing a plurality of operations to implement the program.

An execution screen of the program 130 may be displayed through a display 140. Although the display 140 is illustrated as a separate device connected to the computing device 100 in FIG. 1, the display 140 may be one of the components of the computing device 100 in the case that the computing device 100 is a portable terminal of a user, such as a smartphone, a tablet, and the like. The screen displayed on the display 140 may be a state before information is input to the program or may be an execution result of the program.

Specifically, the processor 110 of the computing device 100 may display a dental image of a patient on the display 140. For example, the dental image may be either a CT image generated by taking a picture of an area including a maxilla, a mandible, and teeth of the patient or a 3D image generated by interpreting the CT image.

In this case, the processor 110 may predict a location of the mandibular nerve of the patient from the dental image by using artificial intelligence (AI). For example, the processor 110 may detect mandibular nerve center points of the patient by analyzing the dental image with AI. In addition, the processor 110 may predict a location of the detected mandibular nerve center points as a location of the mandibular nerve of the patient.

Thereafter, the processor 110 may mark the inferior alveolar nerve on the dental image according to the location of the mandibular nerve predicted from the dental image. Here, the processor 110 may generate the inferior alveolar nerve by connecting the detected mandibular nerve center points and mark the generated inferior alveolar nerve on the dental image. In this case, the inferior alveolar nerve may be a virtual 2D line indicating the predicted location of the mandibular nerve of the patient on the dental image.

Thereafter, the processor 110 may display a rendered 3D tube based on the inferior alveolar nerve. Here, the processor 110 may generate a 3D tube by rendering a tube having a preset radius centered on a mandibular nerve center point included in the inferior alveolar nerve.

In addition, the processor 110 may correct a CT number of an object included in the dental image. The object may be, for example, an anatomical structure such as a bone (or a cortical bone) and a mandibular nerve.

Here, the processor 110 may perform a correction that increases a CT number of an object with a higher CT number than other anatomical structures among objects included in the dental image. In addition, the processor 110 may correct the CT number of an object included in the dental image in proportion to a distance between the object included in the dental image and the inferior alveolar nerve. For example, the processor 110 may increase the CT number of an object included in the dental image by assigning a higher weight to the CT number of the object as the distance between the object and the inferior alveolar nerve increases. On the contrary, the processor 110 may reduce the CT number of an object included in the dental image by assigning a lower weight to the CT number of the object as the distance between the object and the inferior alveolar nerve decreases.

In addition, the processor 110 may set the CT number of an inside of the 3D tube to be lower than the CT number of an outside of the 3D tube.

In this case, the processor 110 may determine a color of an object to be displayed in a color map according to the CT number. For example, the processor 110 may determine a color closer to red as the CT number decreases and may determine a color closer to blue or green as the CT number increases.

In addition, the processor 110 may express the object as a color map on the dental image according to the determined color. Thus, the user may easily identify the distance between an object and an inferior alveolar nerve based on whether the color of the object is closer to red, green, or blue.

That is, the processor 110 may visually clearly distinguish the distance between the inferior alveolar nerve and the object and a boundary between the nerve and the bone by displaying a color map that assigns a weight to an object according to the distance from the mandibular nerve center point based on the mandibular nerve center point of the inferior alveolar nerve.

In addition, since the 3D tube may be a tube having a preset radius centered on the mandibular nerve center point as described above, the inside of the 3D tube in reality may be an inferior alveolar nerve connected to the mandibular nerve center points, and the outside of the 3D tube may be a bone adjacent to the inferior alveolar nerve. Accordingly, the processor 110 may clearly display the boundary between the inferior alveolar nerve and the bone to the user by displaying the inside of the 3D tube and the outside of the 3D tube in different colors according to the CT number.

In addition, during an implant placement, the processor 110 may provide a location guide for the mandibular nerve by marking, on the 3D temporary implant structure, a color indicating the distance between the 3D temporary implant structure and the inferior alveolar nerve according to an implantation depth of the 3D temporary implant structure, wherein the 3D temporary implant structure may indicate an implant body to be placed.

Specifically, the processor 110 may generate a 3D temporary implant structure according to a shape of an implant to be placed in the patient. Thereafter, the processor 110 may place the 3D temporary implant structure in a gum area of the dental image of the patient according to a request of the user. In this case, the processor 110 may correct a CT number of each of 3D temporary implant structure areas in proportion to a distance between the inferior alveolar nerve and each of the 3D temporary implant structure areas that changes during a placement process. In addition, the processor 110 may display a color of each of the 3D temporary implant structure areas according to the corrected CT number.

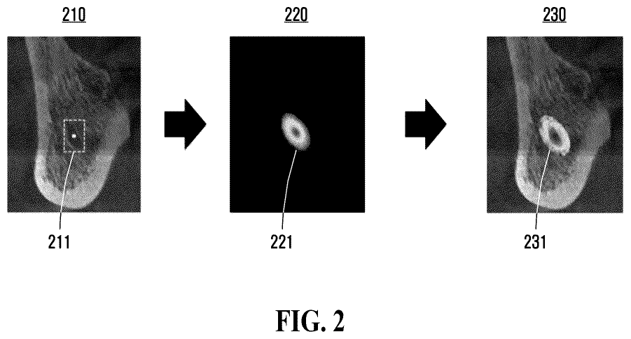

FIG. 2 is an example of an inferior alveolar nerve displaying process according to an embodiment of the present invention.

In operation 210, the processor 110 may detect mandibular nerve center points 211 of the patient from the dental image using AI. In this case, the processor 110 may generate the inferior alveolar nerve by connecting the detected mandibular nerve center points 211 and mark the generated inferior alveolar nerve on the dental image.

In operation 220, the processor 110 may generate a 3D tube 221 from the mandibular nerve center points 211. Specifically, the processor 110 may generate the 3D tube 221 by rendering a tube having a preset radius centered on a mandibular nerve center point included in the inferior alveolar nerve.

In operation 230, the processor 110 may set a CT number of an inside of the 3D tube 221 to be lower than a CT number of an outside of the 3D tube 221. In addition, the processor 110 may clearly display a boundary between the inferior alveolar nerve and a bone to the user by displaying 231 the inside of the 3D tube and the outside of the 3D tube in different colors according to the CT number.

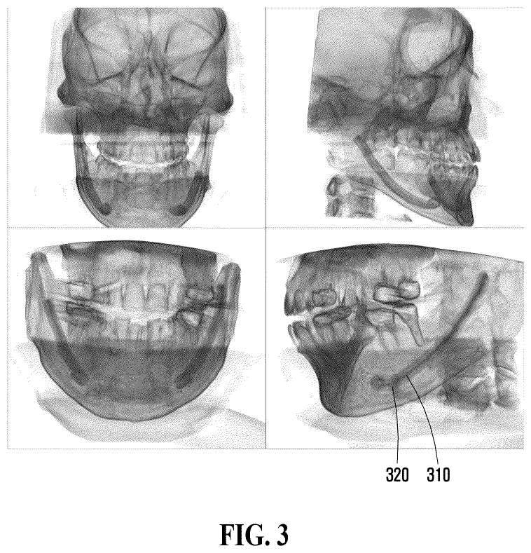

FIG. 3 is an example of an inferior alveolar nerve displayed according to an embodiment of the present invention.

The processor 110 may display the inferior alveolar nerve in a form of a 3D tube with an inside 310 and an outside 320 displayed in different colors, as shown in FIG. 3.

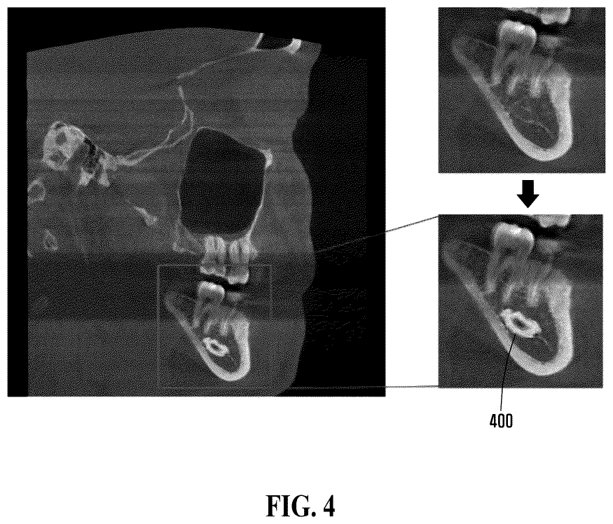

FIG. 4 is an example illustrating a two-dimensional (2D) sagittal section of an inferior alveolar nerve according to an embodiment of the present invention.

The processor 110 may display the inferior alveolar nerve in a form of a 3D tube 400 with an inside and an outside displayed in different colors on a 2D sagittal section of a dental image, as shown in FIG. 4.

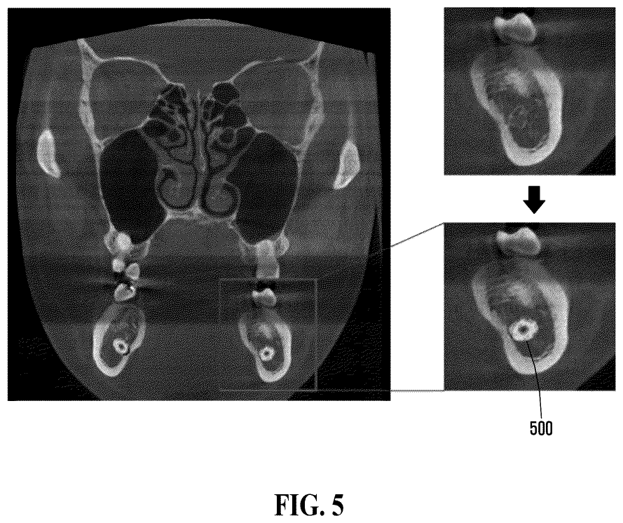

FIG. 5 is an example illustrating a 2D coronal section of an inferior alveolar nerve according to an embodiment of the present invention.

The processor 110 may display the inferior alveolar nerve in a form of a 3D tube 500 with an inside and an outside displayed in different colors on a 2D coronal section of a dental image, as shown in FIG. 5.

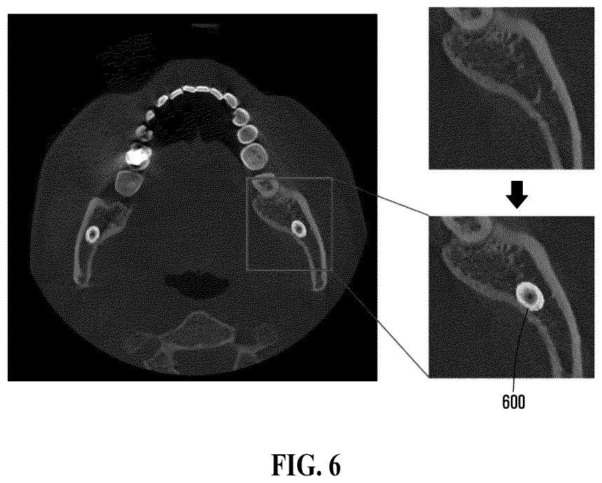

FIG. 6 is an example illustrating a 2D axial cross section of an inferior alveolar nerve according to an embodiment of the present invention.

The processor 110 may display the inferior alveolar nerve in a form of a 3D tube 600 with an inside and an outside displayed in different colors on an axial cross section of a dental image, as shown in FIG. 6.

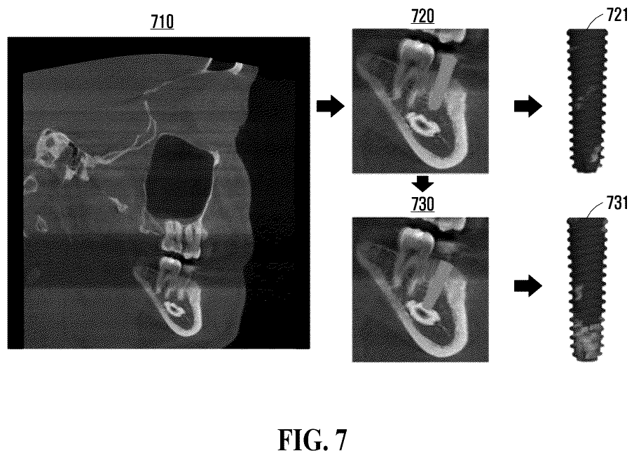

FIG. 7 is an example of guiding to a location of an inferior alveolar nerve during an implant placement, according to an embodiment of the present invention.

In operation 710, the processor 110 may display the inferior alveolar nerve in a form of a 3D tube with an inside and an outside displayed in different colors on a 2D coronal section of a dental image.

In operation 720, the processor 110 may display a 3D temporary implant structure on the dental image according to a request of a user to show a state in which an implant is placed. In this case, the processor 110 may confirm a distance between the 3D temporary implant structure and the inferior alveolar nerve. In addition, when the confirmed distance is greater than or equal to a preset distance, the processor 110 may display an implant structure 721 in a default color.

In operation 730, the processor 110 may change a position of the 3D temporary implant structure according to a request of the user. In this case, the processor 110 may confirm a distance between the 3D temporary implant structure and the inferior alveolar nerve. In addition, when the confirmed distance is less than the preset distance, the processor 110 may correct a CT number of each of 3D temporary implant structure areas in proportion to a distance between each of the 3D temporary implant structure areas and the inferior alveolar nerve. For example, the processor 110 may correct the CT number of an area, among the 3D temporary implant structure areas, which has a distance from the inferior alveolar nerve less than the preset distance, and may maintain the CT number of an area, among the 3D temporary implant structure areas, which has a distance from the inferior alveolar nerve greater than or equal to the preset distance. In addition, the processor 110 may display each of the 3D temporary implant structure areas in a color corresponding to the CT number.

That is, the processor 110 may display 731 in different colors an area of the 3D temporary implant structure having a distance from the inferior alveolar nerve less than the preset distance, as shown in FIG. 7.

That is, the computing device 100 may change a color of the 3D temporary implant structure from the bottom as the 3D temporary implant structure gets closer to the inferior alveolar nerve by an implant placement, thereby allowing the user to easily recognize when the 3D temporary implant structure approaches the inferior alveolar nerve through the color of the 3D temporary implant structure.

In addition, the processor 110 may set different CT numbers for a contact area of the 3D temporary implant structure depending on whether the 3D temporary implant structure contacts a rendered 3D tube based on the inferior alveolar nerve and whether the 3D temporary implant structure contacts the inferior alveolar nerve, and may display a color corresponding to the set CT number.

That is, the processor 110 may determine a CT number to be set for an area of the 3D temporary implant structure according to a condition and may thus display whether the 3D temporary implant structure is in contact with the 3D tube or the inferior alveolar nerve located within the 3D tube through the color of the 3D temporary implant structure.

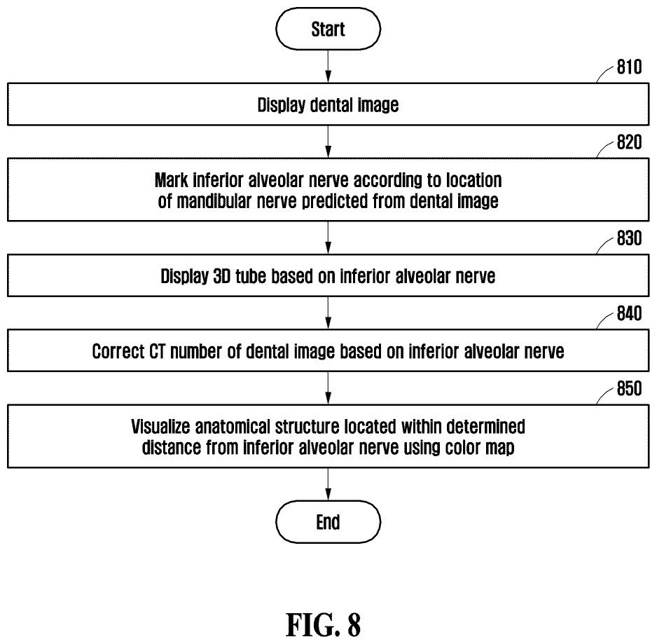

FIG. 8 is a flowchart illustrating an inferior alveolar nerve displaying method according to an embodiment of the present invention.

In operation 810, the processor 110 may display a dental image of a patient on a display 140.

In operation 820, the processor 110 may predict a location of a mandibular nerve of the patient from the dental image using AI. For example, the processor 110 may analyze the dental image to detect the mandibular nerve center points of the patient. In addition, the processor 110 may predict a location of the detected mandibular nerve center points as a location of the mandibular nerve of the patient.

In addition, according to the location of the mandibular nerve predicted from the dental image, the processor 110 may mark the inferior alveolar nerve on a dental image 102. Here, the processor 110 may generate the inferior alveolar nerve by connecting the detected mandibular nerve center points and mark the generated inferior alveolar nerve on the dental image.

In operation 830, the processor 110 may display a rendered 3D tube based on the inferior alveolar nerve marked in operation 820. Here, the processor 110 may generate a 3D tube by rendering a tube having a preset radius centered on a mandibular nerve center point included in the inferior alveolar nerve. For example, the processor 110 may generate a 3D tube having a radius of a voxel value of “30” based on the inferior alveolar nerve. Here, voxel intensity within the 3D tube may have a higher value as a distance between the inferior alveolar nerve located at the center of the 3D tube and a voxel increases. That is, when the voxel intensity of the inferior alveolar nerve is “1,” voxels of the 3D tube may sequentially increase to 2, 3, 4, . . . , starting from a boundary closest to the inferior alveolar nerve, and a boundary of the 3D tube volume may have a voxel value of “30.”

In operation 840, the processor 110 may correct a CT number of an object included in the dental image. For example, the processor 110 may determine CT_{Results}, which may be a corrected CT number of the object according to Equation 1.

$${CT}_{Results} = {CT}_{Origin} \times Tube \qquad [ Equation 1 ]$$

Here, Tube may be equal to a volume size of the dental image (or a CT image). In addition, the processor 110 may determine a Tube value of a remaining area excluding an area in which the 3D tube is displayed in the dental image to be “0.” In addition, the processor 110 may set a Tube value of “1” to “30” for a voxel in the area in which the 3D tube is displayed in the dental image according to the voxel intensity within the 3D tube. Furthermore, CT_{Origin} denotes an original CT image, and the processor 110 may operate the original CT image and Tube as shown in Equation 1 to determine a corrected CT number according to an anatomical structure of a location in which the mandibular nerve is located and a corrected CT image.

In operation 850, the processor 110 may determine a color of an object to be displayed in a color map according to the CT number. In addition, the processor 110 may express the object as a color map on the dental image according to the determined color.

The present invention may express an inferior alveolar nerve line and a surrounding structure through a color map based on a location of the mandibular nerve predicted from the dental image 102.

In addition, during an implant placement, the present invention may provide a location guide for the mandibular nerve by marking, on the 3D temporary implant structure, a color indicating a distance between the inferior alveolar nerve and the 3D temporary implant structure according to an implantation depth of the 3D temporary implant structure, wherein the 3D temporary implant structure may indicate an implant body to be placed.

In addition, the present invention may visually clearly distinguish a boundary between a nerve and a bone by displaying a color map that assigns a weight to an object according to the distance between the mandibular nerve center point and an object such as the bone or an implant structure, based on the mandibular nerve center point.

The inferior alveolar nerve displaying method according to the present invention may be written in a computer-executable program and may be implemented as various recording media such as magnetic storage media, optical reading media, or digital storage media.

The technical ideas of the present disclosure may be implemented as computer-readable code on a non-transitory computer-readable medium. A non-transitory computer-readable storage medium may be, for example, a removable recording medium (e.g., a CD, a DVD, a Blu-ray disc, a USB storage device, or a removable hard disk), a fixed recording medium (e.g., read-only memory (ROM) or random-access memory (RAM)), a hard disk drive (HDD), or a solid-state disk (SSD). A program recorded on a non-transitory computer-readable storage medium may be transmitted to another computing device through a network such as the Internet and installed on the computing device, and may thus be used on the other computing device.

Although the embodiments of the present disclosure have been described with reference to the accompanying drawings, one of ordinary skill in the art to which the present disclosure pertains will understand that the present disclosure may be implemented in other specific forms without changing the technical idea or essential features thereof. Therefore, it should be understood that the embodiments described above are exemplary in all respects and not restrictive. The scope of protection of the present disclosure should be interpreted by the claims below, and all technical ideas within a scope equivalent thereto should be interpreted as being included in the scope of rights of the technical ideas defined by the present disclosure.

1. An inferior alveolar nerve displaying method comprising:
displaying a dental image of a patient;
marking an inferior alveolar nerve on the dental image according to a location of a mandibular nerve predicted from the dental image; and
displaying, on the dental image, a rendered three-dimensional (3D) tube based on the inferior alveolar nerve.

2. The inferior alveolar nerve displaying method of claim 1, wherein the marking of the inferior alveolar nerve comprises:
detecting, using artificial intelligence (AI), mandibular nerve center points from the dental image; and
generating an inferior alveolar nerve by connecting the detected mandibular nerve center points and marking the generated inferior alveolar nerve.

3. The inferior alveolar nerve displaying method of claim 1, wherein the displaying of the 3D tube comprises:
generating the 3D tube by rendering a tube having a preset radius centered on a mandibular nerve center point included in the inferior alveolar nerve.

4. The inferior alveolar nerve displaying method of claim 1, further comprising:
correcting a CT number of an object, which is an anatomical structure included in the dental image.

5. The inferior alveolar nerve displaying method of claim 4, wherein the correcting of the CT number comprises:
correcting the CT number of the object in proportion to a distance between the object and the inferior alveolar nerve.

6. The inferior alveolar nerve displaying method of claim 4, wherein the correcting of the CT number comprises:
setting a CT number of an inside of the inferior alveolar nerve to be lower than a CT number of an outside of the inferior alveolar nerve.

7. The inferior alveolar nerve displaying method of claim 1, further comprising:
marking, on a 3D temporary implant structure, a color indicating a distance between the 3D temporary implant structure and the inferior alveolar nerve according to an implantation depth of the 3D temporary implant structure, wherein the 3D temporary implant structure indicates an implant body to be placed, during an implant placement.

8. The inferior alveolar nerve displaying method of claim 7, wherein the displaying on the 3D temporary implant structure comprises:
placing a 3D temporary implant structure generated according to a shape of an implant to be placed in a patient in a gum area of the dental image of the patient;
correcting a CT number of each of three-dimensional (3D) temporary implant structure areas in proportion to a distance between the inferior alveolar nerve and each of the 3D temporary implant structure areas that changes during a placement process; and
displaying a color of each of the 3D temporary implant structure areas according to the corrected CT number.

9. A non-transitory computer-readable storage medium storing instructions that, when executed by a processor, cause the processor to perform an inferior alveolar nerve displaying method, the method comprising:
displaying a dental image of a patient;
marking an inferior alveolar nerve on the dental image according to a location of a mandibular nerve predicted from the dental image; and
displaying, on the dental image, a rendered three-dimensional (3D) tube based on the inferior alveolar nerve.

10. A computing device comprising:
a processor; and
a memory configured to load or store a program executed by the processor,
wherein the program is configured to perform:
displaying a dental image of a patient,
marking an inferior alveolar nerve on the dental image according to a location of a mandibular nerve predicted from the dental image; and
displaying a three-dimensional (3D) tube based on the inferior alveolar nerve.

11. The computing device of claim 10, wherein the processor is configured to:
detect, using artificial intelligence (AI), mandibular nerve center points from the dental image; and
generate an inferior alveolar nerve by connecting the detected mandibular nerve center points and mark the generated inferior alveolar nerve.

12. The computing device of claim 10, wherein the processor is configured to:
generate the 3D tube by rendering a tube having a preset radius centered on a mandibular nerve center point included in the inferior alveolar nerve.

13. The computing device of claim 10, wherein the processor is configured to mark, on a 3D temporary implant structure, a color indicating a distance between the 3D temporary implant structure and the inferior alveolar nerve according to an implantation depth of the 3D temporary implant structure, wherein the 3D temporary implant structure indicates an implant body to be placed, during an implant placement.
